(19) United States
(12) Patent Application Publication
(10) Pub. No.: US 2006/0276549 A1
(43) Pub. Date: Dec. 7, 2006

(54) Combination and use of drugs

(76) Inventors: Peer B. Jacobson, Libertyville, IL (US); Michael E. Brune, Mundelein, IL (US); Eugene N. Bush, Libertyville, IL (US); Terry J. Opgenorth, Racine, WI (US); Christine A. Collins, Skokie, IL (US); Thomas von Geldern, Richmond, IL (US)

Correspondence Address: LAHIVE & COCKFIELD, LLP;ABBOTT, 28 STATE ST, BOSTON, MA, 02109, US

(21) Appl. No.: 11/432,782

(22) Filed: May 11, 2006

Related U.S. Application Data
(60) Provisional application No. 60/681,096, filed on May 13, 2005.

(51) Int. Cl. A61K 31/137

(52) U.S. Cl. 514/649

(57) ABSTRACT

The present invention is directed to preferred pharmaceutical compositions comprising sibutramine and rimonabant and use of sibutramine and rimonabant to treat obesity and obesity related disorders in a patient.

### RELATED APPLICATIONS

This application claims the benefit of U.S. Provisional Application No. 60/681,096, filed May 13, 2005. The entire contents of this patent application are hereby incorporated herein by reference.

### BACKGROUND OF THE INVENTION

Obesity, which can be defined as a body weight more than 20% above the ideal body weight, is a major health concern in Western societies. Obesity is the result of a positive energy balance, as a consequence of increased ratio of caloric intake to energy expenditure. The molecular factors regulating food intake and body weight balance are incompletely understood. [B. Staels et al., J. Biol. Chem. 270(27), 15958 (1995); F. Lonnquist et al., Nature Medicine 1(9), 950 (1995)]. Although the genetic and/or environmental factors leading to obesity are poorly understood, several genetic factors have been identified.

Obesity causes or exacerbates many health problems, both independently and in association with other diseases. The medical problems associated with obesity, which can be serious and life-threatening, include hypertension; type 2 diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arteriosclerosis; heart disease; abnormal heart rhythms; heart arrythmias (Kopelman, P. G., Nature 404, 635-643 (2000)) and others. The efficacy of currently available weight loss drugs used in monotherapy for the treatment of obesity leave room for improvement. Studies of the weight loss medications orlistat (Davidson, M. H. et al. (1999) JAMA 281:235-42), dexfenfluramine (Guy Grand, B. et al. (1989) Lancet 2:1142-5), sibutramine (Bray, G. A. et al. (1999) Obes. Res. &:189-98) and phentermine (Douglas, A. et al. (1983) Int. J. Obes. 7:591-5) have demonstrated a weight loss of about 5%-10% of body weight for drug compared to placebo. In particular, sibutramine reduces body weight by about 10% over a 6 month or a 1 year period. Studies have also found that sibutramine potently inhibits food intake and decreases body weight initially. Sibutramine is a 5-hydroxytryptamine and noradrenaline reuptake inhibitor in vivo (Buckett, W. R., Thomas, P. C. & Luscombe, G. P. (1988). Prog. Neuro-Psychopharmacol. Biol. Psychiat. 12, 575-584 and Luscombe, G. P., Hopcroft, R. H., Thomas, P. C. & Buckett, W. R. (1989). Neuropharmacology, 28, 129-134.) Studies have shown that it reduces body weight by a dual mode of action; it decreases food intake by enhancing satiety (Fantino, M. & Souquet, A. -M. (1995). Int. J. Obesity, 19, 145; Halford, J. C. G., Heal, D. J. & Blundell, J. E. (1995). Brit. J. Pharmacol. 114, 387P; and Stricker-Krongrad, A., Souquet, A. -M. & Burlet, C. (1995). Int. J. Obesity, 19, 145.), and it increases energy expenditure by stimulating thermogenesis (Connoley, I. P., Heal, D. J. & Stock, M. J. (1995). Brit. J. Pharmacol. 114, 388P; and Connoley, I. P., Frost, I., Heal, D. J. & Stock, M. J. (1996). Brit. J. Pharmacol. 117, 170P).

Another class of weight loss drugs that is currently being studied is the cannabinoid receptor, particularly CB-1, antagonists. Cannabinoid receptor 1 (CB-1) antagonist/inverse agonists are disclosed in: U.S. Pat. Nos. 5,532,237, 4,973,587, 5,013,837, 5,081,122, 5,112,820, 5,292,736, 5,624,941 and U.S. Pat. No. 6,028,084; and PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, and WO 02/076949; and EPO Application No. EP-658546. Specific CB-1 antagonists/inverse agonists useful in the present invention include, but are not limited to, rimonabant (Sanofi Synthelabo), and SR-147778 (Sanofi Synthelabo).

United States Patent Application Number 2004/0122033 discloses that combinations of certain known weight loss drugs may provide more effective treatment of obesity and associated ailments. Included in the vast list of drugs that may be used together are sibutramine and rimonabant. The combination of sibutramine and rimonabant is arrived at by selecting one of many permutations of combinations disclosed therein. Further, it does not specifically disclose the combination of sibutramine and rimonabant and, more importantly, it does not provide any data demonstrating that the combination of sibutramine and rimonabant would have an effect that is more beneficial than taking either agent alone. Further, it does not disclose the most desirable doses of the combination of sibutramine and rimonabant. The data it does show relate to a few different combinations: a combination of a melanocortin receptor agonist and a MCH receptor antagonist; the combination of a CB-1 inverse agonist/antagonist and a NPY antagonist; the combination of a CB-1 inverse agonist/antagonist, a NPY antagonist and a Mc4r agonist; and the combination of a CB-1 inverse agonist/antagonist and a MCH receptor antagonist.

United States Patent Application Number 2005/0032773 discloses that a combination of sibutramine and a CB-1 antagonist exhibited synergistic effect in reducing food consumption. However, the disclosure of 2005/0032773 describes the doses of sibutramine and rimonabant to be used in such a broad range, 0.5 to 10 mg of sibutramine and 0.1 to 200 mg of rimonabant (see paragraph 301) or 1 to 15 mg of sibutramine and 0.1 to 500 mg of rimonabant (see paragraph 303), that the application does not teach, suggest or enable the most efficacious dosage combinations of sibutramine and rimonabant for use in treating obesity (and or other ailments) in humans.

It has now been found that particularly preferred dosage combinations of sibutramine and rimonabant yield synergistic efficacy over the efficacy of either sibutramine or rimonabant monotherapy in treating obesity and other disorders, as detailed herein.

### SUMMARY OF THE INVENTION

The present invention is directed to certain dosage combinations of sibutramine and rimonabant. The present invention is further directed to methods of using the combination or the compounds separately to treat a patient with a disease or condition as described herein.

In one embodiment, the present invention is directed to a pharmaceutical composition comprising between about 5 mg to about 20 mg of sibutramine hydrochloride monohydrate or the mole equivalent amount of a sibutramine compound; between about 2 mg to about 20 mg of rimonabant hydrochloride or the mole equivalent amount of a rimonabant compound; and one or more pharmaceutically suitable carriers or excipients.

In a preferred embodiment, the present invention is directed tp a pharmaceutical composition comprising between about 5 mg to about 19 mg of sibutramine hydrochloride monohydrate or the mole equivalent amount of a sibutramine compound; between about 3 mg to about 20 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising between about 6 mg to about 18 mg of sibutramine hydrochloride monohydrate or the mole equivalent amount of a sibutramine compound; between about 4 mg to about 20 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising between about 7 mg to about 17 mg of sibutramine hydrochloride monohydrate or the mole equivalent amount of a sibutramine compound; between about 5 mg to about 20 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising between about 8 mg to about 16 mg of sibutramine hydrochloride monohydrate or the mole equivalent amount of a sibutramine compound; between about 6 mg to about 20 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising between about 9 mg to about 15 mg of sibutramine hydrochloride monohydrate or the mole equivalent amount of a sibutramine compound; between about 7 mg to about 20 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising between about 10 mg to about 15 mg of sibutramine hydrochloride monohydrate or the mole equivalent amount of a sibutramine compound; between about 10 mg to about 20 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising about 10 mg of sibutramine hydrochloride monohydrate or the mole equivalent amount of a sibutramine compound; about 5 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising about 15 mg of sibutramine hydrochloride monohydrate or the mole equivalent amount of a sibutramine compound; about 5 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising about 10 mg of sibutramine hydrochloride monohydrate or the mole equivalent amount of a sibutramine compound; about 10 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising about 10 mg of sibutramine hydrochloride monohydrate or the mole equivalent amount of a sibutramine compound; about 15 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising about 15 mg of sibutramine hydrochloride monohydrate or the mole equivalent amount of a sibutramine compound; about 10 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising about 15 mg of sibutramine hydrochloride monohydrate or the mole equivalent amount of a sibutramine compound; about 15 mg of rimonabant hydrochloride or the mole equivalent amount of a rimonabant compound; and one or more pharmaceutically suitable carriers or excipients.

In another preferred embodiment, the present invention is directed tp a pharmaceutical composition comprising about 15 mg of sibutramine hydrochloride monohydrate or the mole equivalent amount of a sibutramine compound; about 20 mg of rimonabant hydrochloride or the mole equivalent amount of a rimonabant compound; and one or more pharmaceutically suitable carriers or excipients.

A preferred embodiment of the foregoing is where the pharmaceutical composition is formulated as a tablet, capsule, granules, powders, suspensions or emulsions.

In a further embodiment, the present invention is directed to a method of treating obesity in a patient in need thereof, comprising administering to the patient a first unit dosage of a pharmaceutical composition comprising between about 5 mg to about 20 mg of sibutramine hydrochloride monohydrate or the mole equivalent amount of a sibutramine compound and one or more pharmaceutically suitable carriers or excipients; and administering to the patient a second unit dosage of a pharmaceutical composition comprising between about 2 mg to about 20 mg of rimonabant hydrochloride or the mole equivalent amount of a rimonabant compound; wherein the first unit dosage and the second unit dosage are administered (a) as a single pharmaceutical composition, (b) simultaneously as separate pharmaceutical compositions, (c) sequentially, as separate pharmaceutical compositions starting with the first unit dosage and then administering the second unit dosage or starting with the second unit dosage and then administering the first unit dosage, (d) successively, separated by 1 to 52 weeks, as separate pharmaceutical compositions starting with the first unit dosage and then the second unit dosage or starting with the second unit dosage and then the first unit dosage, or (e) individually followed by combination, wherein administering to the patient with the first unit dosage or the second unit dosage for a period of 1 to 52 weeks and then the other dosage unit is administered to the patient in addition to the dosage unit with which the patient started.

In a preferred embodiment of the method, the present invention is directed tp the method wherein between about 5 mg to about 19 mg of sibutramine hydrochloride monohydrate or the mole equivalent amount of a sibutramine compound is administered; and between about 3 mg to about 20 mg of rimonabant hydrochloride or the mole equivalent amount of a rimonabant compound is administered.

In a preferred embodiment of the method, the present invention is directed tp the method, wherein between about 6 mg to about 18 mg of sibutramine hydrochloride monohydrate or the mole equivalent amount of a sibutramine compound is administered; and between about 5 mg to about 20 mg of rimonabant hydrochloride or the mole equivalent amount of a rimonabant compound is administered.

In a preferred embodiment of the method, the present invention is directed tp the method wherein between about 7 mg to about 17 mg of sibutramine hydrochloride monohydrate or the mole equivalent amount of a sibutramine compound is administered; and between about 7 mg to about 20 mg of rimonabant hydrochloride or the mole equivalent amount of a rimonabant compound is administered.

In a preferred embodiment of the method, the present invention is directed tp the method wherein between about 8 mg to about 16 mg of sibutramine hydrochloride monohydrate or the mole equivalent amount of a sibutramine compound is administered; and between about 9 mg to about 20 mg of rimonabant hydrochloride or the mole equivalent amount of a rimonabant compound is administered.

In a preferred embodiment of the method, the present invention is directed tp the method wherein between about 9 mg to about 15 mg of sibutramine hydrochloride monohydrate or the mole equivalent amount of a sibutramine compound is administered; and between about 10 mg to about 20 mg of rimonabant hydrochloride or the mole equivalent amount of a rimonabant compound is administered.

In a preferred embodiment of the method, the present invention is directed tp the method wherein between about 10 mg to about 15 mg of sibutramine hydrochloride monohydrate or the mole equivalent amount of a sibutramine compound is administered; and between about 12 mg to about 20 mg of rimonabant hydrochloride or the mole equivalent amount of a rimonabant compound is administered.

In a preferred embodiment of the method, the present invention is directed tp the method wherein about 10 mg of sibutramine hydrochloride monohydrate or the mole equivalent amount of a sibutramine compound is administered; and about 5 mg of rimonabant hydrochloride or the mole equivalent amount of a rimonabant compound is administered.

In a preferred embodiment of the method, the present invention is directed tp the method wherein about 15 mg of sibutramine hydrochloride monohydrate or the mole equivalent amount of a sibutramine compound is administered; and about 5 mg of rimonabant hydrochloride or the mole equivalent amount of a rimonabant compound is administered.

In a preferred embodiment of the method, the present invention is directed tp the method wherein about 10 mg of sibutramine hydrochloride monohydrate or the mole equivalent amount of a sibutramine compound is administered; and about 10 mg of rimonabant hydrochloride or the mole equivalent amount of a rimonabant compound is administered.

In a preferred embodiment of the method, the present invention is directed tp the method wherein about 10 mg of sibutramine hydrochloride monohydrate or the mole equivalent amount of a sibutramine compound is administered; and about 15 mg of rimonabant hydrochloride or the mole equivalent amount of a rimonabant compound is administered.

In a preferred embodiment of the method, the present invention is directed tp the method wherein about 15 mg of sibutramine hydrochloride monohydrate or the mole equivalent amount of a sibutramine compound is administered; and about 10 mg of rimonabant hydrochloride or the mole equivalent amount of a rimonabant compound is administered.

In a preferred embodiment of the method, the present invention is directed tp the method wherein about 15 mg of sibutramine hydrochloride monohydrate or the mole equivalent amount of a sibutramine compound is administered; and about 15 mg of rimonabant hydrochloride or the mole equivalent amount of a rimonabant compound is administered.

In a preferred embodiment of the method, the present invention is directed tp the method wherein about 15 mg of sibutramine hydrochloride monohydrate or the mole equivalent amount of a sibutramine compound is administered; and about 20 mg of rimonabant hydrochloride or the mole equivalent amount of a rimonabant compound is administered.

In a preferred method of the foregoing method of treating obesity, the sibutramine hydrochloride monohydrate or the mole equivalent amount of a sibutramine compound and the rimonabant hydrochloride or the mole equivalent amount of a rimonabant compound is administered as a single pharmaceutical composition.

In another preferred method of the foregoing method of treating obesity, the first unit dosage is administered for about 1 to 26 weeks and then the second unit dosage is administered for about 1 to 26 weeks or the second unit dosage is administered for about 1 to 26 weeks and then the first unit dosage is administered for about 1 to 26 weeks.

In each of the foregoing embodiments wherein the method of treating a patient is for treating obesity, the method can also be used to treat one or more of the following diseases or conditions: overeating, bulimia, hypertension, diabetes, elevated plasma insulin concentrations, insulin resistance, dyslipidemias, hyperlipidemia, endometrial cancer, breast cancer, prostate cancer, colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure, e.g, children with acute lymphoblastic leukemia, Metabolic Syndrome (also known as Syndrome X), insulin resistance syndrome, sexual dysfunction, reproductive dysfunction, such as infertility, hypogonadism in males and hirsutism in females, gastrointestinal motility disorders such as obesity-related gastro-esophageal reflux, respiratory disorders such as obesity-hypoventilation syndrome (Pickwickian syndrome), cardiovascular disorders, inflammation such as systemic inflammation of the vasculature, arteriosclerosis, hypercholesterolemia, hyperuricaemia, lower back pain, gallbladder disease, gout, and kidney cancer.

### BRIEF DESCRIPTION OF THE DRAWINGS

FIG. 1A provides the results of the body weight loss observed in the mice of Example 1.

1 B

FIG. 1B provides the results of the caloric intake observed in the mice of Example 1.

FIG. 2A provides the results of the change in glucose in the mice.

FIG. 2B provides the results of the change in glucose in the mice as area under the curve.

FIG. 3A provides the DEXA analysis of the body weight of the mice at Day 28.

FIG. 3B provides the DEXA analysis of the fat mass of the mice at Day 28.

FIG. 3C provides the DEXA analysis of the lean mass of the mice at Day 28.

FIGS. 4A and 4B provide the results of the body weight regain experiment of Example 4.

FIG. 4C provides the results of the suppression of caloric intake of the mice in Example 4.

FIG. 5 provides the results of the snack consumption experiment of Example 5.

FIG. 6 provides an isobologram using the data of Example 5.

FIG. 7 provides the results of the body weight change of the rats used in Example 7.

### DETAILED DESCRIPTION OF THE INVENTION

As used herein, the compound of formula (I) has the formula:

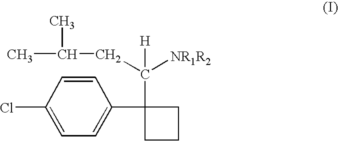

wherein R₁ and R₂ are independently H or methyl, enantiomers, pharmaceutically acceptable salts and pro-drugs thereof. Compounds of formula (I) may also be referred to herein as “sibutramine compounds”. Compounds wherein R₁ and R₂ are each methyl may also be referred to herein as “sibutramine”. The compound of formula (I) wherein R₁ is hydrogen and R₂ is methyl is also known as the Ml metabolite. The compound of formula (I) wherein R₁ and R₂ are each hydrogen is also known as the M2 metabolite.

In the following description of the inventions referring to “a sibutramine compound” or “sibutramine compounds”, the phrases refer to a compound of formula (I) or its pharmaceutically acceptable salts or pro-drugs thereof. The preferred “sibutramine compounds” are sibutramine hydrochloride, sibutramine mesylate, sibutramine hydrochloride monohydrate, sibutramine mesylate hemihydrate, the M1 metabolite and the M2 metabolite.

Sibutramine (Formula (I), R₁═CH₃, R₂═CH₃) has a pharmacological profile which is unique amongst monoamine reuptake inhibitors. Through its pharmacologically active metabolites, (metabolite M1, R₁═H, R₂═CH₃ in Formula (I) and metabolite M2, R₁═H, R₂═H in Formula (I)) sibutramine inhibits the reuptake of all three monoamines differentiating it from serotonin (5-HT)-selective reuptake inhibitors, e.g. fluoxetine, noradrenaline-selective reuptake inhibitors, e.g. desipramine, dopamine-selective reuptake inhibitors, e.g. bupropion, and serotonin-noradrenaline reuptake inhibitors, e.g. venlafaxine. It is this unique combination of pharmacological actions, which renders sibutramine, and the other compounds of formula (I), efficacious in the treatment of obesity and obesity related diseases, such as Metabolic Syndrome.

The sibutramine compounds are a non-selective serotonin and noradrenaline reuptake inhibitor that acts centrally to reduce energy intake by inducing a feeling of fullness (or satiety) after eating and increasing energy expenditure.

Compounds of formula (I) contain a chiral center. When a compound of formula (I) contains a single chiral center it may exist in two enantiomeric forms. The present invention includes the use of the individual enantiomers and mixtures of the enantiomers. The enantiomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer to the other by asymmetric transformation.

The preparation of compounds of formula (I), such as N,N-dimethyl-1-[1-(4-chlorophenyl)cyclobutyl]-3-methylbutylamine and pharmaceutically acceptable salts thereof and the individual enantiomers can be prepared by enantioselective synthesis from optically active precursors, or by resolving the racemic compound which can be prepared as described above. Enantiomers of secondary amines of the formula (I) can also be prepared by preparing the racemate of the corresponding primary amine, resolving the latter into the individual enantiomers, and then converting the optically pure primary amine enantiomer into the required secondary amine by methods described in British Patent Specification 2098602 and U.S. Pat. No. 4,522,828. Specific examples of compounds of formula (I) are: (+)-N-[1-[1-(4-chlorophenyl)cyclobutyl]-3-methylbutyl]-N-methylamine; (−)-N-[1-[1-(4-chlorophenyl)cyclobutyl-3-methylbutyl]-N-methylamine; (+)-1-[1-(4-chlorophenyl)cyclobutyl]-3-methylbutylamine; (−)-1-[1-(4-chlorophenyl)cyclobutyl]-3-methylbutylamine; (+)-N-[1-[1-(4-chlorophenyl)cyclobutyl]-3-methylbutyl]-N,N-dimethylamine; (−)-N-[1-[1-(4-chlorophenyl)cyclobutyl]-3-methylbutyl]-N,N-dimethylamine; (±)-N-[1-[1-(4-chlorophenyl)cyclobutyl]-N-methylamine; (±)-1-[1-(4-chlorophenyl)cyclobutyl]-3-methylbutylamine; (±)-N-[1-[1-(4-chlorophenyl)cyclobutyl]-3-methylbutyl]-N,N-dimethylamine, and mixtures, and pharmaceutically acceptable salts thereof.

Pharmaceutically acceptable salts of a compound of formula (I) include, but are not limited to, hydrochloride, hydrobromide, sulfates, methanesulfonates (mesylate), nitrates, maleates, acetates, citrates, fumarates, tartrates (eg (+)-tartrates (−)-tartrates or mixtures thereof including racemic mixtures], succinates, benzoates and salts with amino acids such as glutamic acid. These salts may be prepared by methods known to those skilled in the art. Preferred salts of sibutramine include hydrochloride and mesylate.

The pharmaceutically acceptable salts of sibutramine may exist as hydrates. The degree of hydration will depend on the salt. The preferred hydrates are sibutramine hydrochloride monohydrate and sibutramine mesylate hemihydrate. The preparation of sibutramine hydrochloride and its monohydrate is described in U.S. Pat. No. 4,929,629. The preparation of sibutramine mesylate and its hemihydrate is described in U.S. patent application Ser. No. 10/678,325, publication no. US 2004/0068018.

Rimonabant is also known as SR-147778 or by the chemical name 1-H-pyrazole-3-carboxamide, 5-(4-chlorophenyl)-1-(2,4-dichlorophenyl)-4-methyl-N-1-piperidinyl and has the structure:

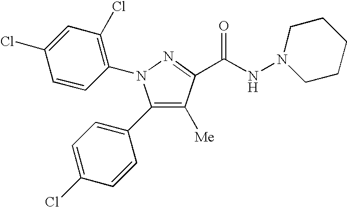

The preferred pharmaceutically acceptable salts of rimonabant are benzenesulfonate, hydrobromide, hydrochloride, citrate, ethanesulfonate, fumarate, gluconate, iodate, isothionate, maleate, methanesulfonate (mesylate), methylenebis-β-oxynaphthoate, nitrate, oxalate, pamoate, phosphate, salicylate, succinate, sulfate, tartrate, theophyllinacetate and p-toluenesulfonate. The preferred salts of rimonabant are the hydrochloride, mesylate and tartrate. The most preferred salt of rimonabant is rimonabant hydrochloride. The phrase “a rimonabant compound” refers to rimonabant or its pharmaceutically acceptable salts or pro-drugs thereof, particularly rimonabant and rimonabant hydrochloride.

Rimonabant and the pharmaceutically acceptable salts thereof can be synthesized according to the procedure described in U.S. Pat. No. 5,624,941.

Whenever a specific milligram (mg) quantity of “sibutramine” is referred to herein in a pharmaceutical composition or a method of the present invention, the amount refers to the amount of sibutramine hydrochloride monohydrate. The specific stated milligram quantities of “rimonabant” referred to herein in a pharmaceutical composition or a method of the present invention refer to the amount of rimonabant hydrochloride. However, the “equivalent mole amount” of another sibutramine compound or rimonabant compound can be used. That is, “the equivalent mole amount” of another sibutramine compound or rimonabant compound can be calculated by one skilled in art by determining the amount of moles of sibutramine hydrochloride monohydrate or rimonabant hydrochloride in the specifically stated amount by dividing the milligram amount by the molecular weight of the appropriate compound. The number of milligrams of the other sibutramine compound or rimonabant compound can then be calculated by taking the molecular weight of the other sibutramine compound or rimonabant compound and multiplying by the molecular weight of the other sibutramine compound or rimonabant compound. For example, if one wished to replace the amount of sibutramine hydrochloride monohydrate with the mole equivalent amount of sibutramine free base, the calculation would be as follows:
10 mg of sibutramine hydrochloride monohydrate divided by the molecular weight of sibutramine hydrochloride monohydrate (334.33 gm/mol) equals 2.99×10⁻⁵ moles of sibutramine hydrochloride monohydrate. Therefore, 2.99×10⁻⁵ moles of sibutramine free base (297.88 gm/mol) equals 8.9 mg of sibutramine free base.

The present invention is the result of a surprising discovery that preferred dosage combinations of a sibutramine compound and a rimonabant compound provided advantages, especially weight loss, when compared to treatment of a patient using either drug alone. Further, the use of preferred dosages of a sibutramine compound and a rimonabant compound provide other advantages. The preferred dosages of each drug in the combination are sub-maximal efficacious doses that together combine to provide effects that may be seen with maximal doses of either drug used alone. Therefore, one of the other advantage of using preferred dosages of a sibutramine compound and a rimonabant compound in combination is that it avoids the deleterious side-effects that would be observed in patients given the maximal dose of either compound alone to achieve similar results as seen with the combination.

Thus, in one aspect, the present invention is directed to pharmaceutical compositions comprising “preferred dosage combinations” of a sibutramine compound and a rimonabant compound and a pharmaceutically acceptable carrier or excipient.

It was surprisingly discovered that administration of preferred amounts of a sibutramine compound and a rimonabant compound in combination resulted in weight loss in Diet Induced Obese (DIO) Mice that was about 50% greater than the weight loss observed in DIO Mice administered with either sibutramine hydrochloride monohydrate or rimonabant hydrochloride alone. The study also demonstrated that the DIO Mice receiving the combination of sibutramine hydrochloride monohydrate and rimonabant hydrochloride exhibited a surprisingly greater decrease in caloric intake than the DIO Mice treated with sibutramine hydrochloride monohydrate or rimonabant hydrochloride alone. As a result of the study, particular dosage combinations of a sibutramine compound and a rimonabant compound have been shown to be surprisingly more efficacious in treating obesity than treating obesity with a sibutramine compound or a rimonabant compound alone.

Further, a measure of insulin sensitivity in the DIO Mice in the study demonstrated that the administration of preferred amounts of a sibutramine compound and a rimonabant compound in combination resulted in improved insulin sensitivity in the DIO Mice compared to the insulin sensitivity observed in the DIO Mice administered with either sibutramine hydrochloride monohydrate or rimonabant hydrochloride alone. Additionally, a separate measure of the DIO Mice in the study revealed that the DIO Mice receiving the combination of sibutramine hydrochloride monohydrate and rimonabant hydrochloride exhibited lower fasting glucose levels compared to the DIO Mice being treated with sibutramine hydrochloride monohydrate or rimonabant hydrochloride alone. As a result of the study, preferred dosage amounts of a sibutramine compound and a rimonabant compound have been shown to be surprisingly more efficacious for improving insulin sensitivity than treating a subject with a sibutramine compound or a rimonabant compound alone.

Another study involving DIO Mice demonstrated that the administration of preferred amounts of sibutramine hydrochloride monohydrate and rimonabant hydrochloride in combination was highly effective in reducing clinically defined snacking when compared to the results in DIO Mice treated with sibutramine hydrochloride monohydrate or rimonabant hydrochloride alone.

Yet another study in DIO Mice demonstrated that administration of preferred amounts of sibutramine hydrochloride monohydrate and rimonabant hydrochloride in combination showed surprisingly better results in treating certain factors that are included in Metabolic Syndrome, thus, indicating the combination will be more effective in treating Metabolic Syndrome when compared to treating Metabolic Syndrome with sibutramine hydrochloride monohydrate or rimonabant hydrochloride alone.

In another embodiment, the present invention is directed to a method of treating obesity in a patient comprising the step of administering preferred dosage ranges or preferred dosage combinations of a sibutramine compound and a rimonabant compound to a patient in need of such treatment.

In another embodiment, the present invention is directed to a method of treating a patient having a disorder associated with excessive caloric intake comprising the step of administering preferred dosage ranges or preferred dosage combinations of a sibutramine compound and a rimonabant compound to a patient in need of such treatment.

In another embodiment, the present invention is directed to a method of maintaining weight in a patient who wishes to keep weight off after having lost weight or not wanting to gain weight comprising the step of administering preferred dosage ranges or preferred dosage combinations of a sibutramine compound and a rimonabant compound to the patient.

In another embodiment, the present invention is directed to a method of treating a patient having an obesity-related disorder comprising the step of administering preferred dosage ranges or preferred dosage combinations of a sibutramine compound and a rimonabant compound to a patient in need of such treatment.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure, e.g, children with acute lymphoblastic leukemia. Further examples of obesity-related disorders are Metabolic Syndrome, also known as Syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compositions of the present invention are also useful for reducing the risk of secondary outcomes of obesity, such as reducing the risk of left ventricular hypertrophy. Therefore, the present invention includes a method of treating each of the foregoing diseases or conditions in a patient with one or more of the diseases or conditions comprising the step of administering to the patient in need of such treatment preferred dosage ranges or preferred dosage combinations of a sibutramine compound and a rimonabant compound.

In another embodiment, the present invention is directed to a method of treating a patient having metabolic syndrome comprising the step of administering to a patient in need of such treatment preferred dosage ranges or preferred dosage combinations of a sibutramine compound and a rimonabant compound.

In another embodiment, the present invention is directed to a method of aiding a patient to quit smoking cigarettes comprising the step of administering to the patient in need of such treatment preferred dosage ranges or preferred dosage combinations of a sibutramine compound and a rimonabant compound. A preferred method of this invention is a method of preventing weight gain in a patient who is quitting smoking or has quit smoking comprising administering to the patient in need of such treatment preferred dosage ranges or preferred dosage combinations of a sibutramine compound and a rimonabant compound.

In another embodiment, the present invention is directed to a method of treating a patient having chronic fatigue syndrome comprising the step of administering to the patient in need of such treatment preferred dosage ranges or preferred dosage combinations of a sibutramine compound and a rimonabant compound.

In another embodiment, the present invention is directed to a method of treating a patient having weight gain associated with the patient taking psychotropic drugs comprising the step of administering to the patient in need of such treatment preferred dosage ranges or preferred dosage combinations of a sibutramine compound and a rimonabant compound.

In another embodiment, the present invention is directed to a method of treating a patient having obsessive-compulsive behavior comprising the step of administering to the patient in need of such treatment preferred dosage ranges or preferred dosage combinations of a sibutramine compound and a rimonabant compound.

In another embodiment, the present invention is directed to a method of treating a patient having pulmonary hypertension comprising the step of administering to the patient in need of such treatment preferred dosage ranges or preferred dosage combinations of a sibutramine compound and a rimonabant compound.

In another embodiment, the present invention is directed to a method of treating menstrual dysfunction in a patient comprising the step of administering to the patient in need of such treatment preferred dosage ranges or preferred dosage combinations of a sibutramine compound and a rimonabant compound.

In another embodiment, the present invention is directed to a method of treating premenstrual syndrome in a patient comprising the step of administering to the patient in need of such treatment preferred dosage ranges or preferred dosage combinations of a sibutramine compound and a rimonabant compound.

In another embodiment, the present invention is directed to a method of treating eating disorders in a patient comprising the step of administering to the patient in need of such treatment preferred dosage ranges or preferred dosage combinations of a sibutramine compound and a rimonabant compound.

In another embodiment, the present invention is directed to a method of treating certain cancers associated with weight gain comprising the step of administering to the patient in need of such treatment preferred dosage ranges or preferred dosage combinations of a sibutramine compound and a rimonabant compound.

In another embodiment, the present invention is directed to a method of treating osteoarthritis or gout in a patient comprising the step of administering to the patient in need of such treatment preferred dosage ranges or preferred dosage combinations of a sibutramine compound and a rimonabant compound.

In another embodiment, the present invention is directed to a method of treating cardiovascular disease in a patient comprising the step of administering to the patient in need of such treatment preferred dosage ranges or preferred dosage combinations of a sibutramine compound and a rimonabant compound.

In another embodiment, the present invention is directed to a method of treating pain, neuropathic pain or fibromyalgia in a patient comprising the step of administering to the patient in need of such treatment preferred dosage ranges or preferred dosage combinations of a sibutramine compound and a rimonabant compound.

In another embodiment, the present invention is directed to a method of treating hyperactivity disorder in a patient comprising the step of administering to the patient in need of such treatment preferred dosage ranges or preferred dosage combinations of a sibutramine compound and a rimonabant compound.

In another embodiment, the present invention is directed to a method of treating a patient having erectile dysfunction comprising the step of administering to the patient in need of such treatment preferred dosage ranges or preferred dosage combinations of a sibutramine compound and a rimonabant compound.

In another embodiment, the present invention is directed to a method of treating hiatial hernias in a patient comprising the step of administering to the patient in need of such treatment preferred dosage ranges or preferred dosage combinations of a sibutramine compound and a rimonabant compound.

In another embodiment, the present invention is directed to a method of treating anxiety disorders in a patient comprising the step of administering to the patient in need of such treatment preferred dosage ranges or preferred dosage combinations of a sibutramine compound and a rimonabant compound.

In another embodiment, the present invention is directed to a method of treating sleep apnea in a patient comprising the step of administering to the patient in need of such treatment preferred dosage ranges or preferred dosage combinations of a sibutramine compound and a rimonabant compound.

In another embodiment, the present invention is directed to a method of improving metabolic rate in a patient comprising the step of administering to the patient in need of such treatment preferred dosage ranges or preferred dosage combinations of a sibutramine compound and a rimonabant compound.

In another embodiment, the present invention is directed to a method of reducing insulin resistance in a patient comprising the step of administering to the patient in need of such treatment preferred dosage ranges or preferred dosage combinations of a sibutramine compound and a rimonabant compound.

What is meant by “preferred dosage ranges” in a method or pharmaceutical composition of the present invention as it applies to sibutramine hydrochloride monohydrate (from which the mole equivalent amount of a sibutramine compound can be calculated) is a dose in the following ranges, inclusive: about 5 mg to about 20 mg; about 5 mg to about 19 mg; about 5 mg to about 18 mg; about 5 mg to about 17 mg; about 5 mg to about 16 mg; about 5 mg to about 15 mg; about 5 mg to about 14 mg; about 5 mg to about 13 mg; about 5 mg to about 12 mg; about 5 mg to about 11 mg; about 5 mg to about 10 mg; about 6 mg to about 20 mg; about 6 mg to about 19 mg; about 6 mg to about 18 mg; about 6 mg to about 17 mg; about 6 mg to about 16 mg; about 6 mg to about 15 mg; about 6 mg to about 14 mg; about 6 mg to about 13 mg; about 6 mg to about 12 mg; about 6 mg to about 11 mg; about 6 mg to about 10 mg; about 7 mg to about 20 mg; about 7 mg to about 19 mg; about 7 mg to about 18 mg; about 7 mg to about 17 mg; about 7 mg to about 16 mg; about 7 mg to about 15 mg; about 7 mg to about 14 mg; about 7 mg to about 13 mg; about 7 mg to about 12 mg; about 7 mg to about 11 mg; about 7 mg to about 10 mg; about 8 mg to about 20 mg; about 8 mg to about 19 mg; about 8 mg to about 18 mg; about 8 mg to about 17 mg; about 8 mg to about 16 mg; about 8 mg to about 15 mg; about 8 mg to about 14 mg; about 8 mg to about 13 mg; about 8 mg to about 12 mg; about 8 mg to about 11 mg; about 8 mg to about 10 mg; about 9 mg to about 20 mg; about 9 mg to about 19 mg; about 9 mg to about 18 mg; about 9 mg to about 17 mg; about 9 mg to about 16 mg; about 9 mg to about 15 mg; about 9 mg to about 14 mg; about 9 mg to about 13 mg; about 9 mg to about 12 mg; about 9 mg to about 11 mg; about 9 mg to about 10 mg; about 10 mg to about 20 mg; about 11 mg to about 19 mg; about 11 mg to about 18 mg; about 11 mg to about 17 mg; about 11 mg to about 16 mg; about 11 mg to about 15 mg; about 11 mg to about 14 mg; about 11 mg to about 13 mg; about 11 mg to about 12 mg; about 12 mg to about 20 mg; about 12 mg to about 19 mg; about 12 mg to about 18 mg; about 12 mg to about 17 mg; about 12 mg to about 16 mg; about 12 mg to about 15 mg; about 12 mg to about 14 mg; about 12 mg to about 13 mg; about 13 mg to about 20 mg; about 13 mg to about 19 mg; about 13 mg to about 18 mg; about 13 mg to about 17 mg; about 13 mg to about 16 mg; about 13 mg to about 15 mg; about 13 mg to about 14 mg; about 14 mg to about 20 mg; about 14 mg to about 19 mg; about 14 mg to about 18 mg; about 14 mg to about 17 mg; about 14 mg to about 16 mg; about 14 mg to about 15 mg; about 15 mg to about 20 mg; about 15 mg to about 19 mg; about 15 mg to about 18 mg; about 15 mg to about 17 mg; about 15 mg to about 16 mg; about 16 mg to about 20 mg; about 16 mg to about 19 mg; about 16 mg to about 18 mg; about 16 mg to about 17 mg; about 17 mg to about 20 mg; about 17 mg to about 19 mg; about 17 mg to about 18 mg; about 18 mg to about 20 mg; about 18 mg to about 19 mg; or about 19 mg to about 20 mg.

What is meant by “preferred dosage ranges” in a method or pharmaceutical composition of the present invention as it applies to rimonabant hydrochloride (from which the mole equivalent amount of a rimonabant compound can be calculated) is a dose in the following ranges, inclusive: about 2 mg to about 20 mg; about 2 mg to about 19 mg; about 2 mg to about 18 mg; about 2 mg to about 17 mg; about 2 mg to about 16 mg; about 2 mg to about 15 mg; about 2 mg to about 14 mg; about 2 mg to about 13 mg; about 2 mg to about 12 mg; about 2 mg to about 11 mg; about 2 mg to about 10 mg; about 3 mg to about 20 mg; about 3 mg to about 19 mg; about 3 mg to about 18 mg; about 3 mg to about 17 mg; about 3 mg to about 16 mg; about 3 mg to about 15 mg; about 3 mg to about 14 mg; about 3 mg to about 13 mg; about 3 mg to about 12 mg; about 3 mg to about 11 mg; about 3 mg to about 10 mg; about 4 mg to about 20 mg; about 4 mg to about 19 mg; about 4 mg to about 18 mg; about 4 mg to about 17 mg; about 4 mg to about 16 mg; about 4 mg to about 15 mg; about 4 mg to about 14 mg; about 4 mg to about 13 mg; about 4 mg to about 12 mg; about 4 mg to about 11 mg; about 4 mg to about 10 mg; about 5 mg to about 20 mg; about 5 mg to about 19 mg; about 5 mg to about 18 mg; about 5 mg to about 17 mg; about 5 mg to about 16 mg; about 5 mg to about 15 mg; about 5 mg to about 14 mg; about 5 mg to about 13 mg; about 5 mg to about 12 mg; about 5 mg to about 11 mg; about 5 mg to about 10 mg; about 6 mg to about 20 mg; about 6 mg to about 19 mg; about 6 mg to about 18 mg; about 6 mg to about 17 mg; about 6 mg to about 16 mg; about 6 mg to about 15 mg; about 6 mg to about 14 mg; about 6 mg to about 13 mg; about 6 mg to about 12 mg; about 6 mg to about 11 mg; about 6 mg to about 10 mg; about 7 mg to about 20 mg; about 7 mg to about 19 mg; about 7 mg to about 18 mg; about 7 mg to about 17 mg; about 7 mg to about 16 mg; about 7 mg to about 15 mg; about 7 mg to about 14 mg; about 7 mg to about 13 mg; about 7 mg to about 12 mg; about 7 mg to about 11 mg; about 7 mg to about 10 mg; about 8 mg to about 20 mg; about 8 mg to about 19 mg; about 8 mg to about 18 mg; about 8 mg to about 17 mg; about 8 mg to about 16 mg; about 8 mg to about 15 mg; about 8 mg to about 14 mg; about 8 mg to about 13 mg; about 8 mg to about 12 mg; about 8 mg to about 11 mg; about 8 mg to about 10 mg; about 9 mg to about 20 mg; about 9 mg to about 19 mg; about 9 mg to about 18 mg; about 9 mg to about 17 mg; about 9 mg to about 16 mg; about 9 mg to about 15 mg; about 9 mg to about 14 mg; about 9 mg to about 13 mg; about 9 mg to about 12 mg; about 9 mg to about 11 mg; about 9 mg to about 10 mg; about 10 mg to about 20 mg; about 11 mg to about 19 mg; about 11 mg to about 18 mg; about 11 mg to about 17 mg; about 11 mg to about 16 mg; about 11 mg to about 15 mg; about 11 mg to about 14 mg; about 11 mg to about 13 mg; about 11 mg to about 12 mg; about 12 mg to about 20 mg; about 12 mg to about 19 mg; about 12 mg to about 18 mg; about 12 mg to about 17 mg; about 12 mg to about 16 mg; about 12 mg to about 15 mg; about 12 mg to about 14 mg; about 12 mg to about 13 mg; about 13 mg to about 20 mg; about 13 mg to about 19 mg; about 13 mg to about 18 mg; about 13 mg to about 17 mg; about 13 mg to about 16 mg; about 13 mg to about 15 mg; about 13 mg to about 14 mg; about 14 mg to about 20 mg; about 14 mg to about 19 mg; about 14 mg to about 18 mg; about 14 mg to about 17 mg; about 14 mg to about 16 mg; about 14 mg to about 15 mg; about 15 mg to about 20 mg; about 15 mg to about 19 mg; about 15 mg to about 18 mg; about 15 mg to about 17 mg; about 15 mg to about 16 mg; about 16 mg to about 20 mg; about 16 mg to about 19 mg; about 16 mg to about 18 mg; about 16 mg to about 17 mg; about 17 mg to about 20 mg; about 17 mg to about 19 mg; about 17 mg to about 18 mg; about 18 mg to about 20 mg; about 18 mg to about 19 mg; or about 19 mg to about 20 mg.

What is meant by “preferred dosage combinations” in a method or pharmaceutical composition of the present invention is the following combination of doses of sibutramine hydrochloride monohydrate and rimonabant hydrochloride. The stated amounts may be adjusted by up to ±2 mg in 0.01 mg increments. As mentioned above, other sibutramine compounds and rimonabant compounds can be used in place of sibutramine hydrochloride monohydrate and rimonabant hydrochloride, respectively, and the milligram dose amounts can be calculated by calculating the equivalent mole amount.

| Dose of sibutramine hydrochloride | Dose of rimonabant |
|---|---|
| monohydrate (mg) | hydrochloride (mg) |
| 5 | 5 |
| 5 | 10 |
| 5 | 15 |
| 5 | 20 |
| 10 | 5 |
| 10 | 10 |
| 10 | 15 |
| 10 | 20 |
| 15 | 5 |
| 15 | 10 |
| 15 | 15 |
| 15 | 20 |

In yet another study, it was surprisingly discovered that treatment of DIO Mice with sibutramine hydrochloride monohydrate alone followed by treatment of the same mice with rimonabant hydrochloride alone resulted in much greater weight loss and much lower caloric intake when compared to DIO Mice that were dosed for the same time period with sibutramine alone or rimonabant alone. As a result of the study, it has been discovered that a method of treating a subject with alternating interval doses of a sibutramine compound and then a rimonabant compound or a rimonabant compound and then a sibutramine compound, results in greater weight loss and a lower total caloric intake during a period of time than a subject that is treated with just a sibutramine compound alone or a rimonabant compound alone for the same period of time. For example, a patient is dosed with a sibutramine compound for 2 months and then the same patient is dosed with a rimonabant compound for 2 months and then the same patient is switched back to a sibutramine compound and so on, alternating back and forth for as long as the patient is being treated to lose weight or maintain a lower weight than when the patient began the treatment regimen or to maintain a desired weight that the patient started at before entering the treatment regimen. The period of time that a patient is treated with a sibutramine compound and the period of time a patient is treated with a rimonabant compound is known as “a cycle”. The cycle does not have to be 2 months for each drug. The cycle can consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or more weeks or 1, 2, 3, 4, 5, 6, 7, 8, 9, 10, 11, 12 or more months for each of the time periods that the patient is taking a sibutramine compound and the time periods that the patient is taking a rimonabant compound, depending on the individual's response to the course of treatment. Further, the cycle does not have to consist of equal time periods for each drug. That is, a sibutramine compound can be administered for 2 months and then a rimonabant compound can be administered for 4 months or vice versa. Such combinations of time intervals within a cycle are encompassed in the scope of the present invention. Furthermore, an attending physician will best be able to determine the most efficacious cycle for an individual in need of treatment.

The preferred time periods of administration of a sibutramine compound and a rimonabant compound in a cycle are to be determined by the patient's physician. For example, a patient is started on a sibutramine compound. The patient is changed over from the sibutramine compound to a rimonabant compound when the physician determines that the patient's weight loss has plateaued on the sibutramine compound. The patient is then kept on the rimonabant compound until the weight loss has plateaued. The physician would then switch the patient back to the sibutramine compound when the patient started to gain weight on the rimonabant compound. The patient would be switched back and forth between the two drugs for as long as the patient is being treated. Preferably, the period that the patient is administered each drug within a cycle is about 2months, 3 months and 4 months.

The doses of sibutramine hydrochloride monohydrate and rimonabant hydrochloride to be used in the foregoing alternating treatment regimen are as follows:
Sibutramine (mg/day)=1, 2, 3, 4, 5, 6, 7, 8, 9, 10, 11, 12, 13, 14, 15, 16,17, 18, 19 or 20. The present invention also encompasses the incremental doses between the foregoing stated numbers by 0.01 mg increments, for example, 10.01 mg, 10.02 mg, 10.03 mg, etc. For the sake of brevity, each incremental number is not written out. However, each incremental number is literally encompassed within the scope of this invention and is explicitly captured by the foregoing statement.
Rimonabant (mg/day)=1, 2, 3, 4, 5, 6, 7, 8, 9, 10, 11, 12, 13, 14, 15, 16, 17, 18, 19 or 20. The present invention also encompasses the incremental doses between the foregoing stated numbers by 0.01 mg increments, for example, 10.01 mg, 10.02 mg, 10.03 mg, etc. For the sake of brevity, each incremental number is not written out. However, each incremental number is literally encompassed within the scope of this invention and is explicitly captured by the foregoing statement.

Preferred doses of sibutramine hydrochloride monohydrate to be used in the alternating dosage regimen as described hereinabove are about 5 mg, 10 mg and 15 mg. More preferred doses of sibutramine hydrochloride monohydrate in the alternating dosage regimen are about 10 mg and 15 mg.

Preferred doses of rimonabant hydrochloride to be used in the alternating dosage regimen as described hereinabove are about 5 mg, 10 mg, 11 mg, 12 mg, 13 mg, 14 mg, 15 mg, 16 mg, 17 mg, 18 mg, 19 mg and 20 mg. More preferred doses of rimonabant hydrochloride in the alternating dosage regimen are 15 mg and 20 mg.

Preferred dosages and time cycles in the alternating dosage regimen comprise selecting a dose of sibutramine hydrochloride monohydrate or the mole equivalent amount of a sibutramine compound in the amount of 10 mg/day, 15 mg/day of 20 mg/day (the stated amounts may be adjusted by up to ±2 mg in 0.01 mg increments); selecting a dose of rimonabant hydrochloride or the mole equivalent amoung of a rimonabant compound in the amount of 10 mg/day, 15 mg/day or 20 mg/day (the stated amounts may be adjusted by up to ±2 mg in 0.01 mg increments); and administering each drug for a time period selected from 1 month, 2 months, 3 months or 4 months. The most preferred doses and time periods of administration are described in the following table.

| Dose of | | Dose of | |
|---|---|---|---|
| Sibutramine | Time on | Rimonabant | Time on |
| (mg/day) | Sibutramine | (mg/day) | Rimonabant |
| 10 | 2 months | 10 | 2 months |
| 10 | 2 months | 15 | 2 months |
| 15 | 2 months | 10 | 2 months |
| 15 | 2 months | 15 | 2 months |
| 15 | 2 months | 20 | 2 months |
| 10 | 3 months | 10 | 3 months |
| 10 | 3 months | 15 | 3 months |
| 15 | 3 months | 10 | 3 months |
| 15 | 3 months | 15 | 3 months |
| 15 | 3 months | 20 | 3 months |
| 10 | 4 months | 10 | 4 months |
| 10 | 4 months | 15 | 4 months |
| 15 | 4 months | 10 | 4 months |
| 15 | 4 months | 15 | 4 months |
| 15 | 4 months | 20 | 4 months |

In another embodiment, the present invention is directed to a kit, which comprises two separate pharmaceutical compositions: a first unit dosage form comprising a prophylactically or therapeutically effective amount of a form of a sibutramine compound, preferably about 5 mg, 10 mg or 15 mg of sibutramine hydrochloride monohydrate, and a pharmaceutically acceptable carrier or diluent, and a second unit dosage form comprising a prophylactically or therapeutically effective amount of a rimonabant compound, preferably 5 mg, 10 mg, 15 mg or 20 mg of rimonabant hydrochloride, and a pharmaceutically acceptable carrier or diluent. The kit further comprises a container. Such kits are preferably suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered. The kit can further include instructions for use to treat, or prevent obesity or one of the obesity related diseases or conditions mentioned herein in a patient in need thereof, such as recommended dosage amounts and when to take the first dosage unit and the second dosage unit. As used herein, the phrase “instructions for use” shall mean any FDA (or its foreign equivalent)-mandated instructions, package inserts, or labels that relate to the administration of the aforementioned compositions or the dosage units in the kit for the purpose of treating obesity or an obesity related disease or disorder, such as metabolic syndrome.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

The term 37 Metabolic Syndrome”, also known as Syndrome X, is associated with a constellation of metabolic abnormalities, which are believed to be associated with insulin resistance or impaired glucose tolerance (Hansen, BC (1999) Ann NY Acad Sci 892:1). Metabolic Syndrome has been recognized as a combination of three or more of the following: abdominal obesity, elevated triglyceride levels, decreased high-density lipoprotein (HDL) cholesterol levels, high blood pressure, and impaired fasting blood glucose, (a measure for decreased insulin sensitivity and increased risk of developing diabetes). (Expert panel on detection evaluation and treatment of high blood cholesterol in adults (2001) JAMA 285:2486-2497). Metabolic Syndrome significantly increases the risk of coronary heart disease (CHD) and atherogenesis, and has been identified as an independent target for coronary heart disease risk reduction, separate from low-density lipoprotein (LDL) cholesterol elevations. Metabolic Syndrome is highly prevalent, with estimates as high as one in four US adults (Ford, E S, et. al. (2002) JAMA 287:356-359). The studies described herein demonstrate that the combination of preferred amounts of sibutramine and rimonabant is more effective in treating Metabolic Syndrome than treating Metabolic Syndrome with either sibutramine or rimonabant alone.

The metabolic derangements associated with obesity have been identified previously, but only recently has Metabolic Syndrome been formally defined and identified as a condition that incurs increased risk for cardiovascular disease, diabetes, and mortality. Treatment guidelines for Metabolic Syndrome focus on weight control and increased physical activity. Weight loss and increased activity are the only intervention to date that is indicated for all of the Metabolic Syndrome components. Pharmacologic therapy for subjects with Metabolic Syndrome has otherwise been disease-specific, i.e. anti-lipid therapy for dyslipidemia and anti-hypertensive therapy for elevated blood pressure. Recently, weight control and increased physical activity have also been identified as the treatment of choice to delay the onset of diabetes mellitus in patients with glucose intolerance (Tuomilehto, J, et. al. (2001) 344:1343-1350; Diabetes Prevention Research Group (2002) N Engl J Med 346:393-403). Treatment of a patient with Metabolic Syndrome with preferred amounts of a sibutramine compound and a rimonabant compound in combination or consecutively is uniquely effective because it positively impacts multiple abnormalities of the syndrome.

The present invention is also directed to a patient who desires to quit smoking. Treatment of such a patient with preferred dosage combinations of sibutramine and rimonabant will result in assisting the patient to quit smoking. A behavior modification program may be used in conjunction with the treatment using a combination of sibutramine and rimonabant. This program may consist of individual counseling sessions or group counseling sessions. As part of the program, nicotine patch may also be applied during the quitting process. The efficacy of the combination of sibutramine and rimonabant to aid a patient to quit smoking may be carried out according to the methods employed by Sanofi-Synthelabo in its STRATUS US trials but using a combination of sibutramine and rimonabant in place of rimonabant alone. To wit, STRATUS-US is a double-blind, placebo-controlled study, conducted in 11 clinical trial sites in the United States. The study enrolled 787 smokers who were motivated to quit smoking, but had previously failed to do so. On average, patients enrolled in the trial were aged 42, smoked 23 cigarettes a day, had been smokers for 11-24 years and were classified as moderately to heavily nicotine-dependent based on the Fagerstrom Scale (measures nicotine dependence). Patients received a daily fixed-dose of either 5 mg or 20 mg of rimonabant, or placebo. Patients were on treatment for 10 weeks. For an initial two-week period, they were allowed to smoke while initiating treatment but were given a target quit date at day 15. Abstinence was determined during the final four weeks of treatment and was measured by carbon monoxide concentrations in expired air (less than or equal to 10 ppm) and by plasma cotinine measurements (the principal nicotine metabolite).

The term “diabetes,” as used herein, includes both insulin-dependent diabetes mellitus (i.e., IDDM, also known as Type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The preferred dosage combinations of sibutramine and rimonabant of the present invention are useful for treating both Type I and Type II diabetes. The combinations of the present invention are also useful for treating and/or preventing gestational diabetes mellitus.

Obesity-induced or obesity-related co-morbidities include, but are not limited to, diabetes, non-insulin dependent diabetes mellitus-type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

“Treatment” or “treating” (of obesity and obesity-related disorders) as used in a method of the present invention refers to the administration of a first unit dosage (either a sibutramine compound or a rimonabant compound) and a second unit dosage (rimonabant if the first unit dosage is sibutramine or sibutramine if the first unit dosage is rimonabant) of the present invention wherein the first unit dosage and the second unit dosage are administered (a) as a single pharmaceutical composition, (b) simultaneously as separate pharmaceutical compositions, (c) sequentially, as separate pharmaceutical compositions starting with the first unit dosage and then administering the second unit dosage, (d) successively, separated by 1 to 52 weeks, as separate pharmaceutical compositions starting with the first unit dosage and then the second unit dosage, or (e) individually followed by combination, wherein starting with the administration to the patient with the first unit dosage for a period of 1 to 52 weeks and then the second unit dosage is administered to the patient in addition to the first unit dosage with which the patient started.

“Prevention” (of obesity and obesity-related disorders) refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the combination of a sibutramine compound and a rimonabant compound of the present invention. Another outcome of prevention may be preventing regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovary disease, cardiovascular diseases, osteoarthritis, dermatological disorders, hypertension, insulin resistance, hypercholesterolemia, hypertriglyceridemia, and cholelithiasis.

The term “subject” and “patient”, are used interchangeably and as used herein refers to an animal, preferably a mammal, most preferably a human, who has been the object of treatment, observation or experiment. In one embodiment the term “mammal” is a “human” said human being either male or female.

The term “subject in need thereof” or “patient in need thereof” refers to a subject or patient who is in need of treatment or prophylaxis as determined by a researcher, veterinarian, medical doctor or other clinician. In one embodiment, a subject in need of treatment is an obese human with or without obesity-related co-morbidities.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the measurement of well-known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system or subject that is being sought by the researcher, veterinarian, medical doctor or other clinician, which includes alleviation of the symptoms of the disorder being treated. The novel methods of treatment of this invention are for disorders known to those skilled in the art.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The prodrug may also have improved solubility in pharmacological compositions over the parent drug. Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

The pharmaceutical compositions of the present invention comprise a combination of a sibutramine compound and a rimonabant compound and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compounds suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. These compositions may be conveniently presented in unit dosage form and prepared by any of the methods well-known in the art of pharmacy.

For administration by inhalation, the compositions of the present invention are conveniently delivered in the form of an aerosol spray presentation from pressurized packs or nebulizers. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients.

Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will, of course be continuous rather than intermittent throughout the dosage regimen.

The compositions of the present invention can also be administered in the form of liposome delivery systems, such as small unilamellar vesicles, large unilamellar vesicles and multilamellar vesicles. Liposomes can be formed from a variety of phospholipids, such as cholesterol, sterylamine or phosphatidylcholines.

Compositions of the present invention may also be delivered by the use of monoclonal antibodies as individual carriers to which the compound molecules are coupled. The compounds in these compositions may also be coupled with soluble polymers as targetable drug carriers. Such polymers can include polyvinylpyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

Compositions of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

In practical use, each compound in the compositions of the present invention (e.g. each appetite suppressant, each metabolic rate enhancer, each nutrient absorption inhibitor) can be combined as the active ingredients in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredients, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil.

The contents of all of the publications, published patent applications and patents listed herein are incorporated herein by reference in their entirety.

In order to illustrate the invention, the following examples are included. These examples do not limit the invention. They are only meant to suggest a method of reducing the invention to practice and are presented for illustrative purposes.

### EXAMPLE 1

Body Weight Loss in Diet-induced Obese (DIO) Mice

C57BL/6J mice (age 5-6 weeks) were obtained from Jackson Labs (Bar Harbor, Me.) and housed in groups of 5 under conditions of 12 hours lights on, 12 hours lights off (on at 04:00 h), with food and water available ad libitum. At the beginning of the study, mice were administered a purified low fat diet (D12450Bi, 10 kcal % fat, 3.8 kcal/g) or a high fat content diet (D12492i, 60 kcal % fat, 5.2 kcal/g), both obtained from Research Diets Inc. (New Brunswick, N.J.) for approximately 16 weeks. The fat content of these diets was a mixture of lard and soybean oil.

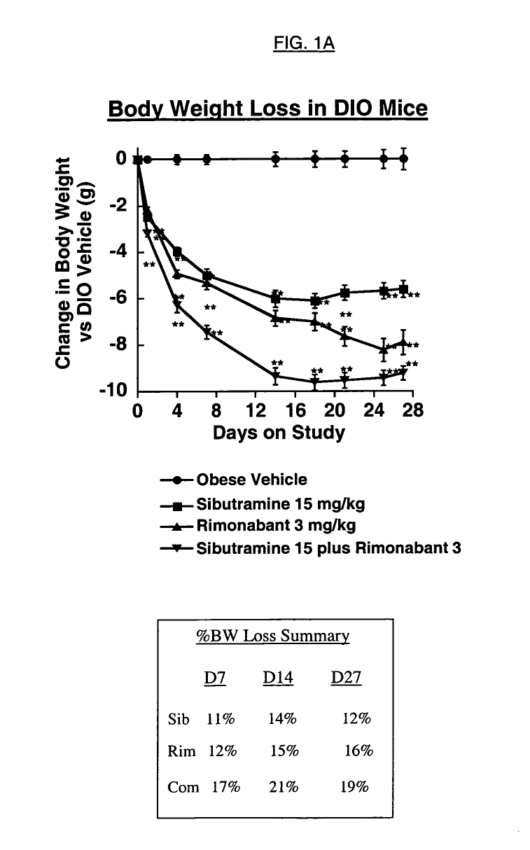

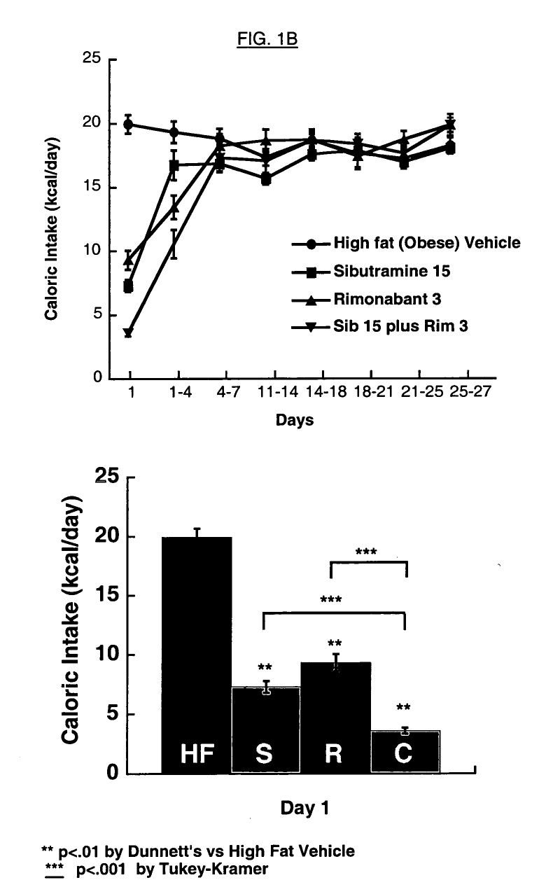

Mice were individually housed for study two weeks prior to start of treatment. Mice were weighed one week later (experiment day -7), and conditioned to oral gavage and daily vehicle administration by daily oral treatment at 15:00 h with Tween-80 (Sigma Chemical, St. Louis, Mo.) 1% vehicle in water (V/V). All doses were given in 4 ml/kg body weight volume of vehicle. All compound doses are expressed as base equivalent weights per unit body weight. Mice were reweighed on day -1 to ensure that weight loss plateaued and was not progressive. Mice were stratified into groups of equal mean body weight, with n=20 mice per group. On days 0 to 28, mice were dosed by oral gavage at 15:00 h (q.d.). Sibutramine hydrochloride monohydrate and rimonabant hydrochloride were administered p.o. by gavage for 27 to 28 days at 15:00 h, at doses of 15 mg/kg and 3 mg/kg base, respectively, once a day (q.d.). Compounds were formulated in Tween-80 vehicle. For combination treatment, the individual compound formulations were mixed together immediately prior to dosing, and given as a single gavage. Food and body weights were determined on days 0 prior to treatment, and on days 1, 4, 7, 11, 14, 18, 21, 25, 27 and 28. Ten mice per group were fasted overnight on day 27. Tail blood was obtained on day 28 of study for blood glucose determination (Precision Glucometer, Abbott Labs), then sacrificed by CO₂ asphyxiation and exsanguinations via cardiac puncture. Plasma was prepared with K₃-EDTA anticoagulant by centrifugation, and stored at −80° C. Plasma was analysed for insulin, leptin, free fatty acids, triglycerides, total cholesterol, HDLc and LDLc. HOMA index of insulin resistance was determined from the fasting glucose and insulin from the equation: HOMA IR=((fasting glucose (mg/dl)*fasting insulin(ng/ml)*25.05)/405. Body fat and lean content were determined by dual energy X-ray absorptiometry (DEXA, Piximus II, GE-Lunar Corp). Subsequent to DEXA scan, fat pads, liver, and kidneys were weighed, and a 100 mg segment of liver was saved at −20° C. for subsequent measurement of liver TG concentration. Results are shown in FIGS. 1A and 1B. The data in FIG. 1A demonstrate that while both sibutramine and rimonabant reduce body weight nearly equally out to day 14, the combination of the drugs significantly improves weight loss efficacy at day 14 (21% vs 14% and 15%, respectively). From Day 14-28, sibutramine's effects plateau in this rodent model of obesity, and the rimonabant treated group continues to lose approximately 4% more than the sibutramine treated animals. However, mice treated with the combination of sibutramine and rimonabant continue to show greater weight loss at the end of 4 weeks vs. either treatment alone. FIG. 1B demonstrates that caloric intake during the first few days of therapy is more significantly reduced in the combination group vs. individual treatments with sibutramine or rimonabant.

### EXAMPLE 2

Insulin Tolerance Test (ITT):

The other n=10 DIO and lean mice per treatment group were treated until day 29, and fasted for 4 hours. Fasting blood glucose was determined by tail blood, then insulin (0.25 U/kg Humulin-R, Eli Lilly) was given intraperitoneally in 10 ml/kg of sterile saline containing 0.1% bovine serum albumin carrier. Tail blood glucose was determined at 30, 60, 90 and 120 min after the insulin injection. Changes in blood glucose over time were summarized in an area under the curve, with units of glucose change (mg/dl)*minutes.

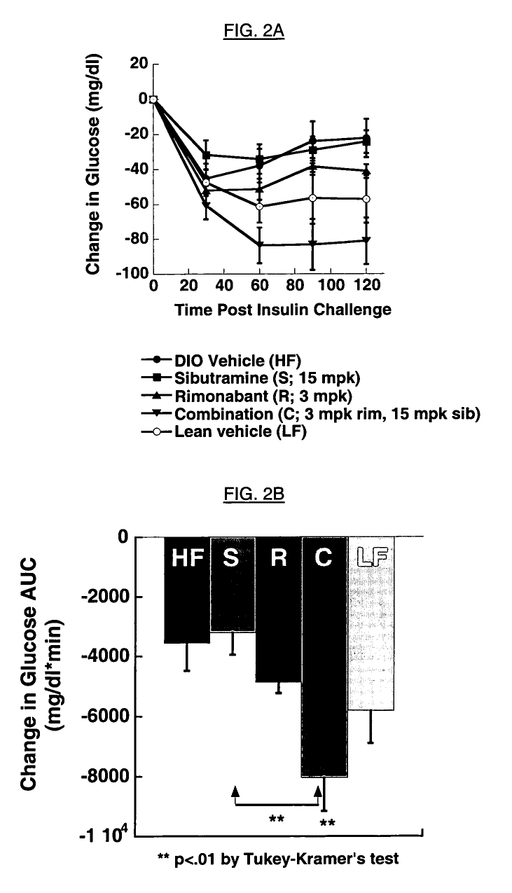

Results are shown in FIGS. 2A and 2B, which demonstrate that treatment of DIO mice for 4 weeks with a combination of sibutramine and rimonabant significantly improves insulin sensitivity vs. either treatment alone. The response with combination treatment is unexpectedly better than the lean control animals, which due to their age have mild insulin resistance as well. The time course data is summarized in the area under the curve analysis in FIG. 2B.

### EXAMPLE 3

DEXA Analysis of Body Composition in DIO Mice

Dual Energy X-Ray Absorptiometry (DEXA) was performed (n=3-6 mice) immediately post-mortem with a GE Lunar PixiMus II densitometer for determination of % adipose and lean tissue.

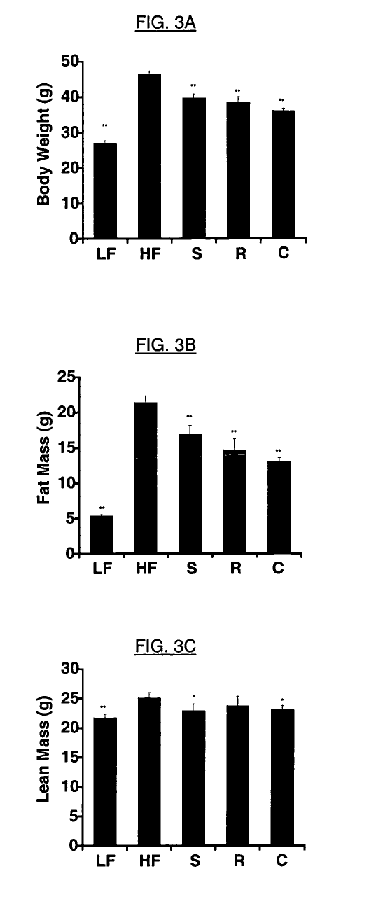

Results are shown in FIGS. 3A, 3B and 3C, which are DEXA image analysis of the mice at Day 28 at the end of the study. FIGS. 3A, 3B and 3C show that all treatment groups significantly decreased body weight and total body fat mass, but that the combination group was more effective than either treatment alone on these parameters. There were statistical decreases in lean mass vs. DIO controls with sibutramine and the combination groups, but these small changes are not viewed as physiologically relevant in comparison to the significant loss in fat mass.

### EXAMPLE 4

Prevention of Body Weight Re-Gain in Diet-induced Obese (DIO) Mice

C57BL/6J mice (age 5-6 weeks) were obtained from Jackson Labs (Bar Harbor, Me.) and housed in groups of 5 under conditions of 12 hours lights on, 12 hours lights off (on at 04:00 h), with food and water available ad libitum. At the beginning of the study, mice were administered a purified low fat diet (D12450Bi, 10 kcal % fat, 3.8 kcal/g) or a high fat content diet (D12492i, 60 kcal % fat, 5.2 kcal/g), both obtained from Research Diets Inc. (New Brunswick, N.J.) for approximately 16 weeks. The fat content of these diets was a mixture of lard and soybean oil. Groups of mice that had been on the high fat diet were switched back to the low fat diet for approximately 4 weeks prior to their being re-introduced to either the high fat food (weight re-gain) or maintained on low fat food. Approximately 5 days prior to the onset of dosing study (Day -5), mice were acclimated to handling and conditioned to oral gavage and daily vehicle administration by daily oral treatment at 15:00 h with Tween-80 (Sigma Chemical, St. Louis, Mo.) 1% vehicle in water (VN). All doses were given in 4 ml/kg body weight volume of vehicle. At the start of drug treatment, mice were dosed qd at 15:00 hr each day for 7 days, and body weights and food intake were evaluated.

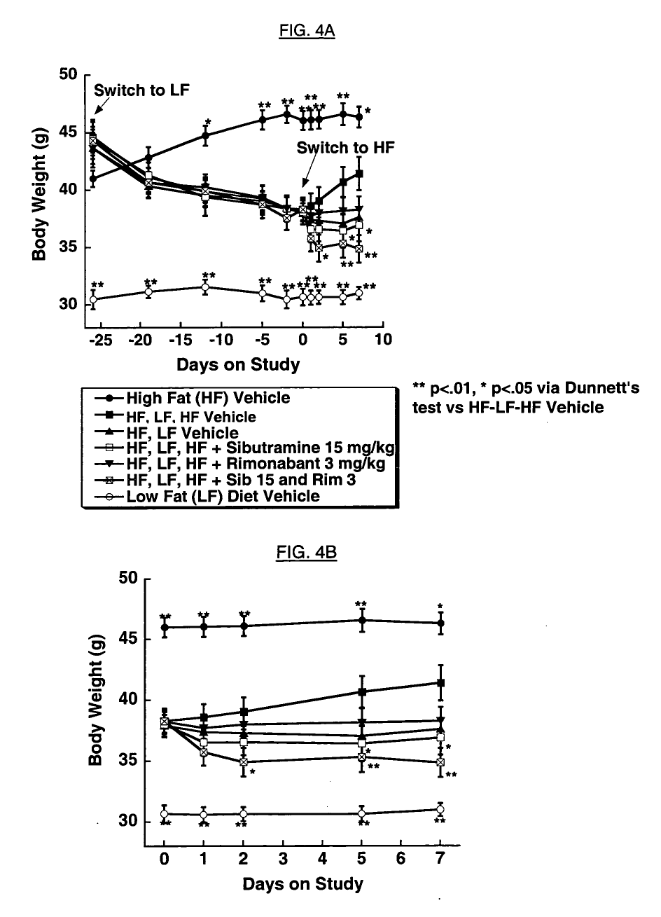

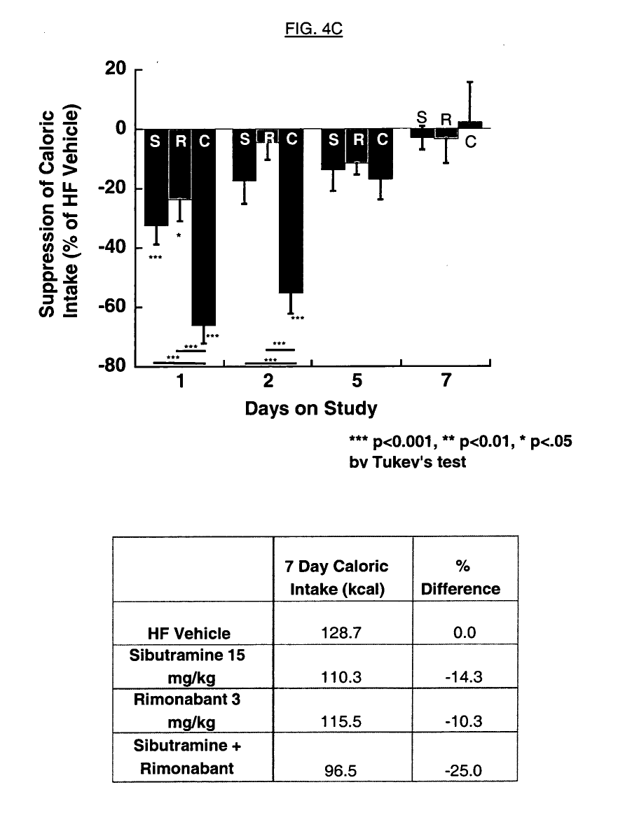

Results are shown in FIGS. 4A, 4B and 4C. FIGS. 4A and 4B demonstrate that while both sibutramine and rimonabant prevent body weight re-gain (day 0-7) vs the HF, LF, to HF control (solid squares), the combination group had a greater initial rate of body weight loss, and continued to lose weight compared to animals treated with either sibutramine or rimonabant alone which simply maintained body weight at the HF to LF vehicle control level (upright solid triangle). FIG. 4C demonstrates the effects of these treatments on caloric intake, and that there is a pronounced reduction on this parameter in the combination treatment group vs. either sibutramine or rimonabant alone.

### EXAMPLE 5

Rat Model of Snacking Behavior

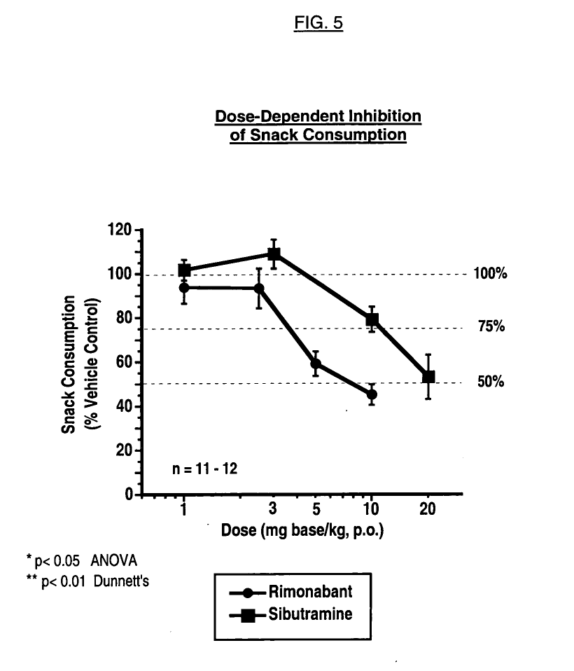

Non-fasted male Zucker fa/fa −/− rats (450 gr) were habituated for 3 weeks to a twice weekly, 3 hour exposure to 60% fat diet (RDI #12492) achieving an average snack intake of 12 to 16 g. Compounds or vehicle (1% Tween 80/water) were dosed orally, 1 ml/kg, n=12-15/group, 1 hour before exclusive presentation of the snack. Snack intake and subsequent 21-hour intake of standard chow (Purina 5008) were measured. Results are shown in FIG. 5, which data indicate that both sibutramine and rimonabant are effective in reducing snack intake in Zucker fa/fa rats, with respective ED50s of 24 and 8 mg/kg, respectively. It is unclear from these individual studies, however, if combination treatment would result in an even greater effect on snacking behavior as reflected in a shift of the ED50 to lower doses. Therefore, an isobologram analysis was performed as described in Example 6

### EXAMPLE 6

Isobologram Analysis for Combination Study Analysis:

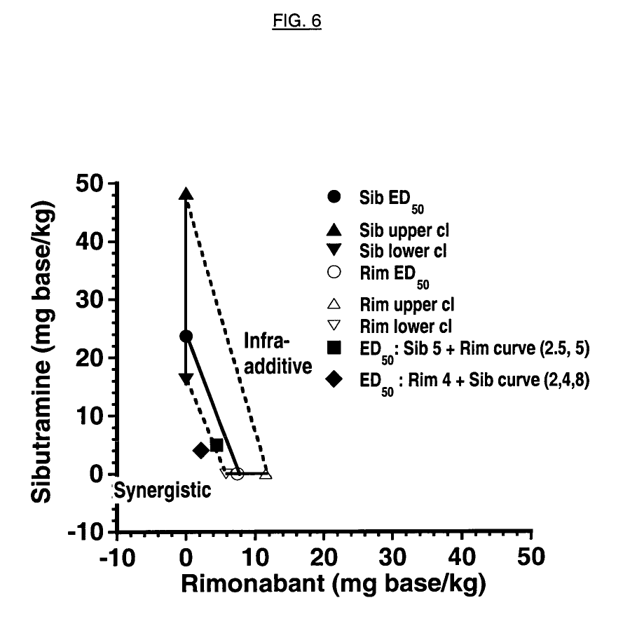

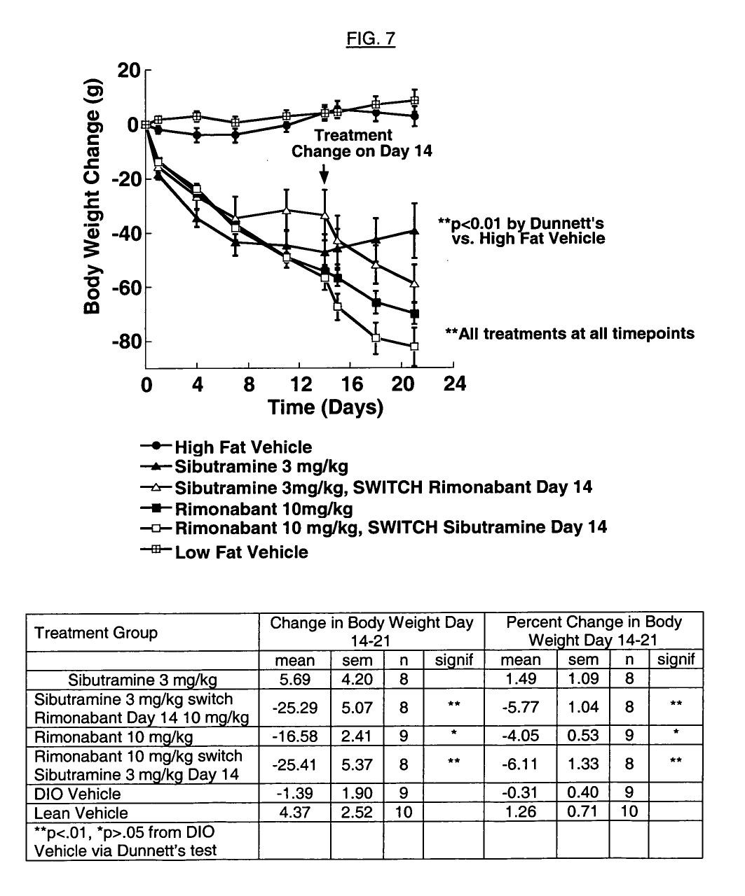

Dose response curves for sibutramine hydrochloride monohydrate and rimonabant hydrochloride were generated in the rat snacking model and ED₅₀ and 95% confidence limits derived from the linear part of the curve by linear regression. Two combination studies in snacking (n=12 rats/group) were performed; the first with a fixed dose of sibutramine HCl (5 mg base/kg) co-administered with rimonabant at 1, 2.5, and 5 mg base/kg, and the second with rimonabant fixed at 4 mg base/kg with sibutramine at 2, 4, and 8 mg base/kg, plus vehicle control. ED50 values were derived from the combination studies and plotted on the isobologram. The ED50 is the dose where there is a 50% reduction in Snack Consumption. FIG. 6 shows that the ED50 for sibutramine hydrochloride monohydrate alone is 24 mg/kg. The rimonabant hydrochloride ED50 is 8 mg/kg. The combination of sibutramine hydrochloride monohydrate and rimonabant hydrochloride, achieves an ED50 for inhibition using significantly, proportionately less sibutramine hydrochloride monohydrate and rimonabant hydrochloride.

### EXAMPLE 7

Weight Loss in Diet-Induced Obese Rats, and Effects of Treatment Switch on Body Weight

Female Sprague-Dawley rats were obtained from Charles River Labs (Raleigh, N.C.) at 5-6 weeks age, and were placed on an ad libitum high fat diet (RDI D020403 Marette's diet (40 kcal % fat; high)) for approximately 30 weeks, resulting in excess body weight gain, or low fat diet (PMI 5001 Research Diets, Inc). Vehicle (1% Tween-80 in water, dose volume 2 ml/kg), sibutramine (3 mpk/day), rimonabant (10 mg/kg/day) were given by daily oral gavage at approximately 15:00 h, for 14 days. On Day 14, half the rats that had been dosed with 3 mpk/day of sibutramine were switched to 10 mpk rimonabant between days 14-21; the remaining sibutramine rats were maintained on that drug as a control. Similarly, half the rats previously dosed with rimonabant were switched to 3 mpk/day of sibutramine. Body weight and food intake were determined daily.

Statistics for DIO Rats:

The statistical significance of body weight, food intake and other parameters was evaluated with Graphpad Instat (GraphPad Software, San Diego Calif.). A one-way analysis of variance was run first, if this was significant then an appropriate multiple comparisons test was used. To determine whether an effect was different from a vehicle control only, Dunnell's test was used, whereas to determine whether responses to compounds were different from each other then Tukey-Kramer's test was used.

Results are shown in FIG. 7, where the graph shows that after 14 days of treatment, when rats were switched to the other drug, there was a significant and unexpected decrease in both total body weight and rate of body weight loss vs. the original treatments.

What is claimed is:

1. A pharmaceutical composition comprising
between about 5 mg to about 20 mg of sibutramine hydrochloride monohydrate or the mole equivalent amount of a sibutramine compound;
between about 2 mg to about 20 mg of rimonabant hydrochloride or the mole equivalent amount of a rimonabant compound; and
one or more pharmaceutically suitable carriers or excipients.

2. The pharmaceutical composition according to claim 1 comprising
between about 5 mg to about 19 mg of sibutramine hydrochloride monohydrate or the mole equivalent amount of a sibutramine compound;
between about 3 mg to about 20 mg of rimonabant hydrochloride or the mole equivalent amount of a rimonabant compound; and
one or more pharmaceutically suitable carriers or excipients.

3. The pharmaceutical composition according to claim 1 comprising
between about 6 mg to about 18 mg of sibutramine hydrochloride monohydrate or the mole equivalent amount of a sibutramine compound;
between about 4 mg to about 20 mg of rimonabant hydrochloride or the mole equivalent amount of a rimonabant compound; and
one or more pharmaceutically suitable carriers or excipients.

4. The pharmaceutical composition according to claim 1 comprising
between about 7 mg to about 17 mg of sibutramine hydrochloride monohydrate or the mole equivalent amount of a sibutramine compound;
between about 5 mg to about 20 mg of rimonabant hydrochloride or the mole equivalent amount of a rimonabant compound; and
one or more pharmaceutically suitable carriers or excipients.

5. The pharmaceutical composition according to claim 1 comprising
between about 8 mg to about 16 mg of sibutramine hydrochloride monohydrate or the mole equivalent amount of a sibutramine compound;
between about 6 mg to about 20 mg of rimonabant hydrochloride or the mole equivalent amount of a rimonabant compound; and
one or more pharmaceutically suitable carriers or excipients.

6. The pharmaceutical composition according to claim 1 comprising
between about 9 mg to about 15 mg of sibutramine hydrochloride monohydrate or the mole equivalent amount of a sibutramine compound;
between about 7 mg to about 20 mg of rimonabant hydrochloride or the mole equivalent amount of a rimonabant compound; and
one or more pharmaceutically suitable carriers or excipients.

7. The pharmaceutical composition according to claim 1 comprising
between about 10 mg to about 15 mg of sibutramine hydrochloride monohydrate or the mole equivalent amount of a sibutramine compound;
between about 10 mg to about 20 mg of rimonabant hydrochloride or the mole equivalent amount of a rimonabant compound; and
one or more pharmaceutically suitable carriers or excipients.

8. The pharmaceutical composition according to claim 1 comprising
about 10 mg of sibutramine hydrochloride monohydrate or the mole equivalent amount of a sibutramine compound;
about 5 mg of rimonabant hydrochloride or the mole equivalent amount of a rimonabant compound; and
one or more pharmaceutically suitable carriers or excipients.

9. The pharmaceutical composition according to claim 1 comprising
about 15 mg of sibutramine hydrochloride monohydrate or the mole equivalent amount of a sibutramine compound;
about 5 mg of rimonabant hydrochloride or the mole equivalent amount of a rimonabant compound; and
one or more pharmaceutically suitable carriers or excipients.

10. The pharmaceutical composition according to claim 1 comprising
about 10 mg of sibutramine hydrochloride monohydrate or the mole equivalent amount of a sibutramine compound;
about 10 mg of rimonabant hydrochloride or the mole equivalent amount of a rimonabant compound; and
one or more pharmaceutically suitable carriers or excipients.

11. The pharmaceutical composition according to claim 1 comprising
about 10 mg of sibutramine hydrochloride monohydrate or the mole equivalent amount of a sibutramine compound;
about 15 mg of rimonabant hydrochloride or the mole equivalent amount of a rimonabant compound; and
one or more pharmaceutically suitable carriers or excipients.

12. The pharmaceutical composition according to claim 1 comprising
about 15 mg of sibutramine hydrochloride monohydrate or the mole equivalent amount of a sibutramine compound;
about 10 mg of rimonabant hydrochloride or the mole equivalent amount of a rimonabant compound; and
one or more pharmaceutically suitable carriers or excipients.

13. The pharmaceutical composition according to claim 1 comprising
about 15 mg of sibutramine hydrochloride monohydrate or the mole equivalent amount of a sibutramine compound;
about 15 mg of rimonabant hydrochloride or the mole equivalent amount of a rimonabant compound; and
one or more pharmaceutically suitable carriers or excipients.

14. The pharmaceutical composition according to claim 1 comprising
about 15 mg of sibutramine hydrochloride monohydrate or the mole equivalent amount of a sibutramine compound;
about 20 mg of rimonabant hydrochloride or the mole equivalent amount of a rimonabant compound; and
one or more pharmaceutically suitable carriers or excipients.

15. The pharmaceutical composition according to claim 1 formulated as a tablet, capsule, granules, powders, suspensions or emulsions.

16. A method of treating obesity in a patient in need thereof, comprising
administering to the patient a first unit dosage of a pharmaceutical composition comprising between about 5 mg to about 20 mg of sibutramine hydrochloride monohydrate or the mole equivalent amount of a sibutramine compound and one or more pharmaceutically suitable carriers or excipients; and
administering to the patient a second unit dosage of a pharmaceutical composition comprising between about 2 mg to about 20 mg of rimonabant hydrochloride or the mole equivalent amount of a rimonabant compound; wherein the first unit dosage and the second unit dosage are administered (a) as a single pharmaceutical composition, (b) simultaneously as separate pharmaceutical compositions, (c) sequentially, as separate pharmaceutical compositions starting with the first unit dosage and then administering the second unit dosage or starting with the second unit dosage and then administering the first unit dosage, (d) successively, separated by 1 to 52 weeks, as separate pharmaceutical compositions starting with the first unit dosage and then the second unit dosage or starting with the second unit dosage and then the first unit dosage, or (e) individually followed by combination, wherein
administering to the patient with the first unit dosage or the second unit dosage for a period of 1 to 52 weeks and then the other dosage unit is administered to the patient in addition to the dosage unit with which the patient started.

17. A method according to claim 16, wherein between about 5 mg to about 19 mg of sibutramine hydrochloride monohydrate or the mole equivalent amount of a sibutramine compound is administered; and
between about 3 mg to about 20 mg of rimonabant hydrochloride or the mole equivalent amount of a rimonabant compound is administered.

18. A method according to claim 16, wherein between about 6 mg to about 18 mg of sibutramine hydrochloride monohydrate or the mole equivalent amount of a sibutramine compound is administered; and
between about 5 mg to about 20 mg of rimonabant hydrochloride or the mole equivalent amount of a rimonabant compound is administered.

19. A method according to claim 16 wherein between about 7 mg to about 17 mg of sibutramine hydrochloride monohydrate or the mole equivalent amount of a sibutramine compound is administered; and
between about 7 mg to about 20 mg of rimonabant hydrochloride or the mole equivalent amount of a rimonabant compound is administered.

20. A method according to claim 16 wherein between about 8 mg to about 16 mg of sibutramine hydrochloride monohydrate or the mole equivalent amount of a sibutramine compound is administered; and
between about 9 mg to about 20 mg of rimonabant hydrochloride or the mole equivalent amount of a rimonabant compound is administered.

21. A method according to claim 16 wherein between about 9 mg to about 15 mg of sibutramine hydrochloride monohydrate or the mole equivalent amount of a sibutramine compound is administered; and
between about 10 mg to about 20 mg of rimonabant hydrochloride or the mole equivalent amount of a rimonabant compound is administered.

22. A method according to claim 16 wherein between about 10 mg to about 15 mg of sibutramine hydrochloride monohydrate or the mole equivalent amount of a sibutramine compound is administered; and
between about 12 mg to about 20 mg of rimonabant hydrochloride or the mole equivalent amount of a rimonabant compound is administered.

23. A method according to claim 16 wherein about 10 mg of sibutramine hydrochloride monohydrate or the mole equivalent amount of a sibutramine compound is administered; and
about 5 mg of rimonabant hydrochloride or the mole equivalent amount of a rimonabant compound is administered.

24. A method according to claim 16 wherein about 15 mg of sibutramine hydrochloride monohydrate or the mole equivalent amount of a sibutramine compound is administered; and
about 5 mg of rimonabant hydrochloride or the mole equivalent amount of a rimonabant compound is administered.

25. A method according to claim 16 wherein about 10 mg of sibutramine hydrochloride monohydrate or the mole equivalent amount of a sibutramine compound is administered; and
about 10 mg of rimonabant hydrochloride or the mole equivalent amount of a rimonabant compound is administered.

26. A method according to claim 16 wherein about 10 mg of sibutramine hydrochloride monohydrate or the mole equivalent amount of a sibutramine compound is administered; and
about 15 mg of rimonabant hydrochloride or the mole equivalent amount of a rimonabant compound is administered.

27. A method according to claim 16 wherein about 15 mg of sibutramine hydrochloride monohydrate or the mole equivalent amount of a sibutramine compound is administered; and
about 10 mg of rimonabant hydrochloride or the mole equivalent amount of a rimonabant compound is administered.

28. A method according to claim 16 wherein about 15 mg of sibutramine hydrochloride monohydrate or the mole equivalent amount of a sibutramine compound is administered; and
about 15 mg of rimonabant hydrochloride or the mole equivalent amount of a rimonabant compound is administered.

29. A method according to claim 16 wherein about 15 mg of sibutramine hydrochloride monohydrate or the mole equivalent amount of a sibutramine compound is administered; and
about 20 mg of rimonabant hydrochloride or the mole equivalent amount of a rimonabant compound is administered.

30. The method according to claim 16 wherein the sibutramine hydrochloride monohydrate or the mole equivalent amount of a sibutramine compound and the rimonabant hydrochloride or the mole equivalent amount of a rimonabant compound is administered as a single pharmaceutical composition.

31. The method according to claim 16 wherein the first unit dosage is administered for about 1 to 26 weeks and then the second unit dosage is administered for about 1 to 26 weeks or the second unit dosage is administered for about 1 to 26 weeks and then the first unit dosage is administered for about 1 to 26 weeks.
